(19) United States
(12) Patent Application Publication
(10) Pub. No.: US 2018/0184947 A1
(43) Pub. Date: Jul. 5, 2018

(54) Integrated Goniometry System and Method for Use of Same

(71) Applicant: Physmodo, Inc., Plano, TX (US)

(72) Inventors: Skylar George Richards, Little Elm, TX (US); Andrew Menter, Dallas, TX (US); Mohammad Almoyyad, Denton, TX (US); Anastasios Chrysanthopoulos, Frisco, TX (US); Randall Joseph Paulin, Los Angeles, CA (US); Nake A. Sekander, Plano, TX (US); David Espenlaub, Plano, TX (US)

(21) Appl. No.: 15/860,019

(22) Filed: Jan. 2, 2018

Related U.S. Application Data
(60) Provisional application No. 62/440,838, filed on Dec. 30, 2016.

(51) Int. Cl. A61B 5/11; A61B 5/00; A61B 5/107

(52) U.S. Cl. CPC A61B 5/1121; A61B 5/0077; A61B 5/1128; A61B 5/4528; A61B 5/486; A61B 2505/09; A61B 5/0075; A61B 5/743; A61B 5/0062; A61B 5/1071; A61B 2576/00; A61B 5/4884

(57) ABSTRACT

An integrated goniometry system and method for use of the same are disclosed. In one embodiment of the goniometry system, an optical sensing instrument, a display, a processor, and memory are communicatively interconnected within a busing architecture in a housing. The optical sensing instrument monitors a stage, which is a virtual volumetric cubic area that is compatible with human exercise positions and movement. The display faces the stage and includes an interactive portal which provides prompts, including an exercise movement prompt providing instructions for a user on the stage to execute a set number of repetitions of an exercise movement, such as a bodyweight overhead squat. The optical sensing instrument senses body point data of the user during each exercise movement. Based on the sensed body point data, a mobility score, an activation score, a posture score, and a symmetry score may be calculated.

### PRIORITY STATEMENT & CROSS-REFERENCE TO RELATED APPLICATIONS

This application claims priority from co-pending United States Patent Application No. 62/440,838, entitled “System and Method for Measuring and Analyzing Physiological Deficiency and Providing Corrective Therapeutic Exercises” and filed on Dec. 30, 2016, in the names of Skylar George Richards et al.; which is hereby incorporated by reference for all purposes.

### TECHNICAL FIELD OF THE INVENTION

The present disclosure relates, in general, to biomechanical evaluations and assessments, which are commonly referred to as range of motion assessments, and more particularly, to automating a biomechanical evaluation process, including a range of motion assessment, and providing recommended exercises to improve physiological inefficiencies of a user.

### BACKGROUND OF THE INVENTION

Human beings have regularly undergone physical examinations by professionals to assess and diagnose their health issues. Healthcare history has been predominantly reactive to an adverse disease, injury, condition or symptom. Increasingly, in modern times, with more access to information, a preventative approach to healthcare has been gaining greater acceptance. Musculoskeletal health overwhelmingly represents the largest health care cost. Generally speaking, a musculoskeletal system of a person may include a system of muscles, tendons and ligaments, bones and joints, and associated tissues that move the body and help maintain the physical structure and form. Health of a person's musculoskeletal system may be defined as the absence of disease or illness within all of the parts of this system. When pain arises in the muscles, bones, or other tissues, it may be a result of either a sudden incident (e.g., acute pain) or an ongoing condition (e.g., chronic pain). A healthy musculoskeletal system of a person is crucial to health in other body systems, and for overall happiness and quality of life. Musculoskeletal analysis, or the ability to move within certain ranges (e.g., joint movement) freely and with no pain, is therefore receiving greater attention. However, musculoskeletal analysis has historically been a subjective science, open to interpretation of the healthcare professional or the person seeking care.

In 1995, after years of research, two movement specialists, Gray Cook and Lee Burton, attempted to improve communication and develop a tool to improve objectivity and increase collaboration efforts in the evaluation of musculoskeletal health. Their system, the Functional Movement Screen (FMS), is a series of 7 different movement types, measured and graded on a scale of 0-3. While their approach did find some success in bringing about a more unified approach to movement assessments, the subjectivity, time restraint and reliance on a trained and accredited professional to perform the evaluation limited its adoption. Accordingly, there is a need for improved systems and methods for measuring and analyzing physiological deficiency of a person and providing corrective recommended exercises while minimizing the subjectivity during a musculoskeletal analysis.

### SUMMARY OF THE INVENTION

It would be advantageous to achieve systems and methods that would improve upon existing limitations in functionality with respect to measuring and analyzing physiological deficiency of a person. It would also be desirable to enable a computer-based electronics and software solution that would provide enhanced goniometry serving as a basis for furnishing corrective recommended exercises while minimizing the subjectivity during a musculoskeletal analysis. To better address one or more of these concerns, an integrated goniometry system and method for use of the same are disclosed. In one embodiment of the integrated goniometry system, an optical sensing instrument, a display, a processor, and memory are communicatively interconnected within a busing architecture in a housing. The optical sensing instrument monitors a stage, which is a virtual volumetric cubic area that is compatible with human exercise positions and movement. The display faces the stage and includes an interactive portal which provides prompts, including an exercise movement prompt providing instructions for a user on the stage to execute a set number of repetitions of an exercise movement, such as a bodyweight overhead squat. The optical sensing instrument senses body point data of the user during each exercise movement. Based on the sensed body point data, a mobility score, an activation score, a posture score, and a symmetry score may be calculated. A composite score may also be calculated. One or more of the calculated scores may provide the basis for determining recommended exercises. These and other aspects of the invention will be apparent from and elucidated with reference to the embodiments described hereinafter.

### BRIEF DESCRIPTION OF THE DRAWINGS

For a more complete understanding of the features and advantages of the present invention, reference is now made to the detailed description of the invention along with the accompanying figures in which corresponding numerals in the different figures refer to corresponding parts and in which:

FIG. 1A is a schematic diagram depicting one embodiment of an integrated goniometry system for measuring and analyzing physiological deficiency of a person, such as a user, and providing corrective recommended exercises according to an exemplary aspect of the teachings presented herein;

FIG. 1B is a schematic diagram depicting one embodiment of the integrated goniometry system illustrated in FIG. 1A, wherein a user from a crowd has approached the integrated goniometry system;

FIG. 2A is an illustration depicting one embodiment of an interactive portal generated by the integrated goniometry system, which is initiating a screening process for automated biomechanical movement assessment of a user;

FIG. 2B is an illustration depicting one embodiment of the interactive portal generated by the integrated goniometry system, which is conducting a screening process for automated biomechanical movement assessment of a user;

FIG. 2C is an illustration depicting one embodiment of an interactive portal generated by the integrated goniometry system, which is conducting a screening process for automated biomechanical movement assessment of a user;

FIG. 2D is an illustration depicting one embodiment of an interactive portal generated by the integrated goniometry system, which is concluding a screening process for automated biomechanical movement assessment of a user;

FIG. 2E is an illustration depicting one embodiment of an interactive portal generated by the integrated goniometry system, which is providing analysis following the screening process for automated biomechanical movement assessment of a user;

FIG. 2F is an illustration depicting one embodiment of an interactive portal generated by the integrated goniometry system, which is concluding a screening process for automated biomechanical movement assessment of a user;

FIG. 3A is a schematic diagram depicting one embodiment of the integrated goniometry system of FIG. 1 within an on-property deployment;

FIG. 3B is a schematic diagram depicting one embodiment of the integrated goniometry system of FIG. 1 within a cloud-based computing deployment serving multiple sites;

FIG. 4A is an illustration of a human skeleton;

FIG. 4B is an illustration of one embodiment of body point data captured by the integrated goniometry system;

FIG. 5 is diagram depicting one embodiment of a set number of repetitions which are monitored and captured by the integrated goniometry system;

FIG. 6 is a functional block diagram depicting one embodiment of the integrated goniometry system presented in FIGS. 3A and 3B;

FIG. 7 is a functional block diagram depicting one embodiment of a server presented in FIGS. 3A and 3B;

FIG. 8 is a conceptual module diagram depicting a software architecture of an integrated goniometry application of some embodiments;

FIG. 9 is a flow chart depicting one embodiment of a method for integrated goniometric analysis according to exemplary aspects of the teachings presented herein; and

FIG. 10 is a flow chart depicting one embodiment of a method implemented in a computing device for measuring and analyzing physiological deficiency of a person and providing corrective recommended exercises according to exemplary aspects of the teachings presented herein.

### DETAILED DESCRIPTION OF THE INVENTION

While the making and using of various embodiments of the present invention are discussed in detail below, it should be appreciated that the present invention provides many applicable inventive concepts, which can be embodied in a wide variety of specific contexts. The specific embodiments discussed herein are merely illustrative of specific ways to make and use the invention, and do not delimit the scope of the present invention.

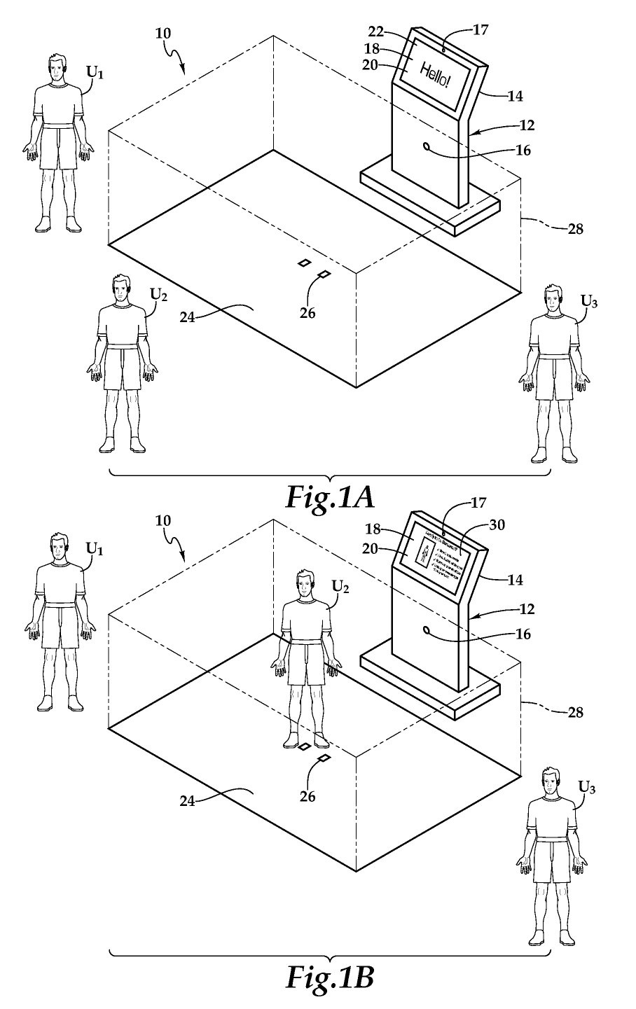

Referring initially to FIG. 1A, therein is depicted one embodiment of an integrated goniometry system for performing automated biomechanical movement assessments, which is schematically illustrated and designated 10. As shown, the system 10 includes an integrated goniometer 12 having a housing 14 securing an optical sensing instrument 16 and a display 18. The display includes an interactive portal 20 which provides prompts, such as a welcoming prompt 22, which may greet a crowd of potential users U₁, U₂, and U₃ and invite a user to enter a stage 24, which may include markers 26 for foot placement of a user standing at the markers 26 to utilize the integrated goniometry system 10. The stage 24 may be a virtual volumetric cubic area 28 that is compatible with human exercise positions and movement. The display 18 faces the stage 24 and the optical sensing instrument 16 monitors the stage 24. A webcam 17 may be included in some embodiments. It should be appreciated that the location of the optical sensing instrument 16 and the webcam 17 may vary with the housing 14. Moreover, the number of optical sensing instruments used may vary also. Multiple optical sensing instruments may be employed. It should be appreciated that the design and presentation of the integrated goniometer 12 may vary depending on application.

Referring now to FIG. 1B, a user, user U₂, has entered the stage 24 and the interactive portal 20 includes an exercise movement prompt 30 providing instructions for the user U₂ on the stage 24 to execute a set number of repetitions of an exercise movement, such as a squat or a bodyweight overhead squat, for example. A series of prompts on the interactive portal 20 instruct the user U₂ while the optical sensing instrument 16 senses body point data of the user U₂ during each exercise movement. Based on the sensed body point data, a mobility score, an activation score, a posture score, a symmetry score, or any combination thereof, for example, may be calculated. A composite score may also be calculated. One or more of the calculated scores may provide the basis for the integrated goniometry system 10 determining an exercise recommendation.

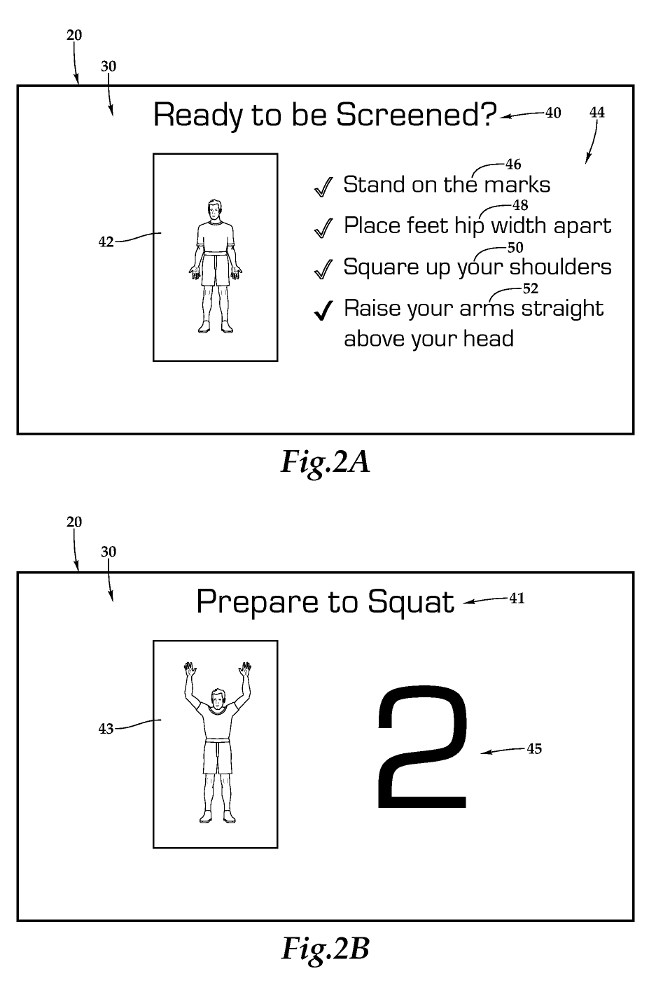

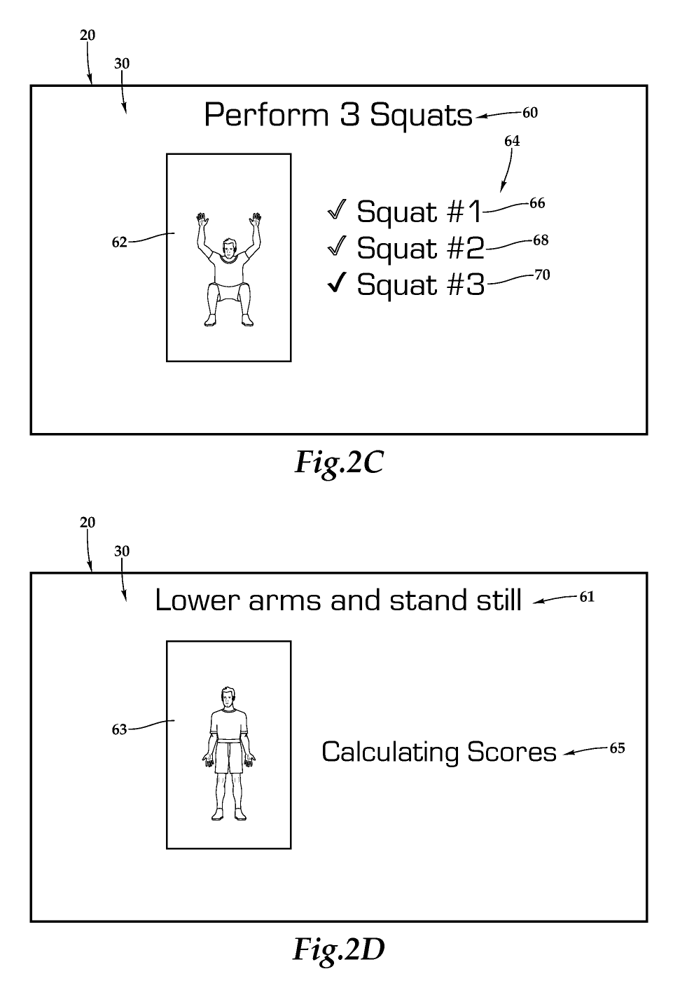

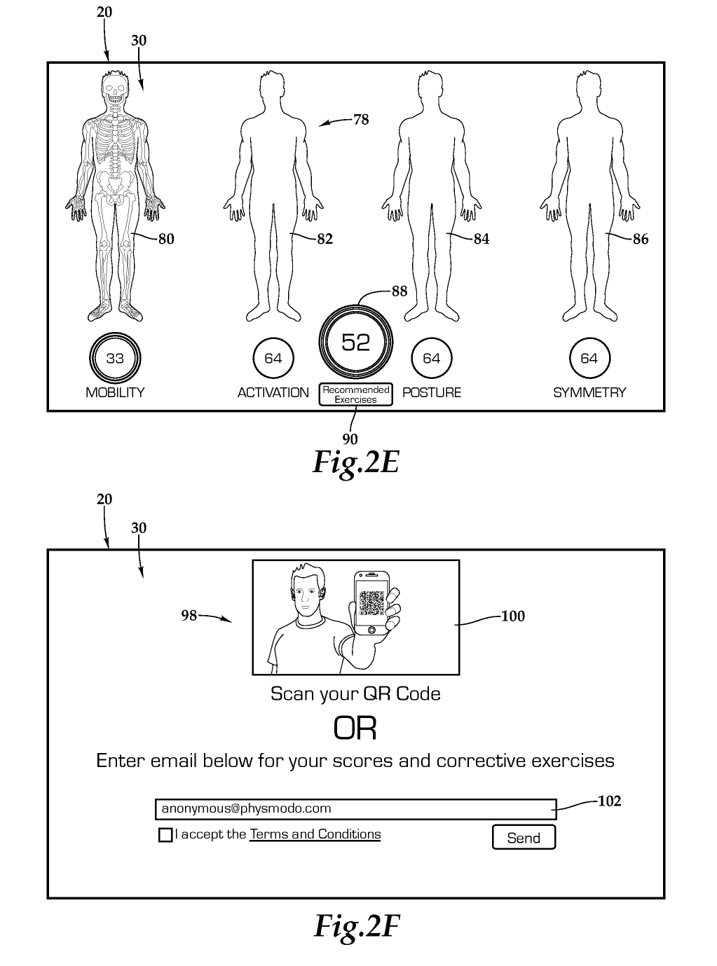

As mentioned, a series of prompts on the interactive portal 20 instruct the user U₂ through repetitions of exercise movements while the optical sensing instrument 16 senses body point data of the user U₂. FIGS. 2A through 2D depict exemplary prompts. FIG. 2A displays the interactive portal 20 including the exercise movement prompt 30 having a visual depiction 42 of the exercise movement. As shown, in one embodiment, the visual depiction may include a front elevation view of a model user performing the exercise movement in an ideal fashion. The visual depiction of the model user may be static or dynamic. In other embodiments, a side elevation view or other view of the model user may be employed. In further embodiments, multiple views, such as a front elevation view and a side elevation view may be shown of the model user. In still further embodiments, the visual depiction of the model user performing the exercise movement is accompanied by a substantially-real time image or video of the user performing the exercise. With a side-by-side presentation of the ideal exercise movement and the user performing the exercise, the user is able to evaluate and self-correct. The exercise movement prompt 30 includes an announcement 40 and checkmarks 44 as progress points 46, 48, 50, 52 confirming the body of the user is aligned properly with the optical sensing instrument such that joint positions and key movements may be accurately measured. FIG. 2B displays the interactive portal 20 with an exercise prepare prompt 41 providing instructions for the user to stand in the exercise start position with a visual depiction 43 of the exercise start position. A countdown for the start of the exercise is shown at counter 45. FIG. 2C displays the interactive portal 20 including an exercise movement prompt 60 having a visual depiction 62 of the exercise movement, such as, for example, a squat, and checkmarks 64 as repetition counts 66, 68, 70 mark progress by the user through the repetitions. FIG. 2D displays the interactive portal 20 including an exercise end prompt 61 providing instructions for the user to stand in an exercise end position as shown by a visual depiction 63 with information presentation 65 indicating the next step that will be undertaken by the integrated goniometry system 10.

Referring now to FIG. 2E, following the completion of the repetitions of the exercise movement, as shown by the score prompt 78, a mobility body map and score 80, an activation body map and score 82, a posture body map and score 84, and a symmetry body map and score 86 may be calculated and displayed. As shown, the mobility body map and score 80 is selected, and the body map portion of the mobility body map and score 80 may show an indicator or heat map of various inefficiencies related to the mobility score. Other body map and scores may have a similar presentation. Further, a composite score 88 may be displayed as well as corrective recommended exercises generated by the integrated goniometry system based on an individual's physiological inefficiencies. As illustrated in the interactive portal, recommended exercises 90 may be accessed and include a number of “foundational” exercises, which may address the primary musculoskeletal issues detected. In one embodiment, these foundational exercises may be determined by consulting an exercise database either locally (e.g., an exercise database stored in the storage 234 of the integrated goniometer 12) or externally (e.g., an external exercise database stored in the storage 254 of the server 110). In one aspect, the foundational exercises determined for each user may not change for a period of time (e.g., several weeks) so as to allow physiological changes of the user to occur. The user may also receive several variable exercises that change daily to promote variability in isolation or supplementary exercises. For example, the user may be instructed to watch videos detailing how to perform these exercises as well as mark them as completed. The user may re-evaluate on a routine basis to check progress and achieve more optimal physiological changes. FIG. 2F shows the interactive portal 20 at the completion of the automated biomechanical movement assessment where a registration and verification prompt 98 includes QR code scanning capability 100 and email interface 102. It should be appreciated that the design and order of the exercise prompts depicted and described in FIG. 2A through FIG. 2F is exemplary. More or less exercise prompts may be included. Additionally, the order of the exercise prompts may vary.

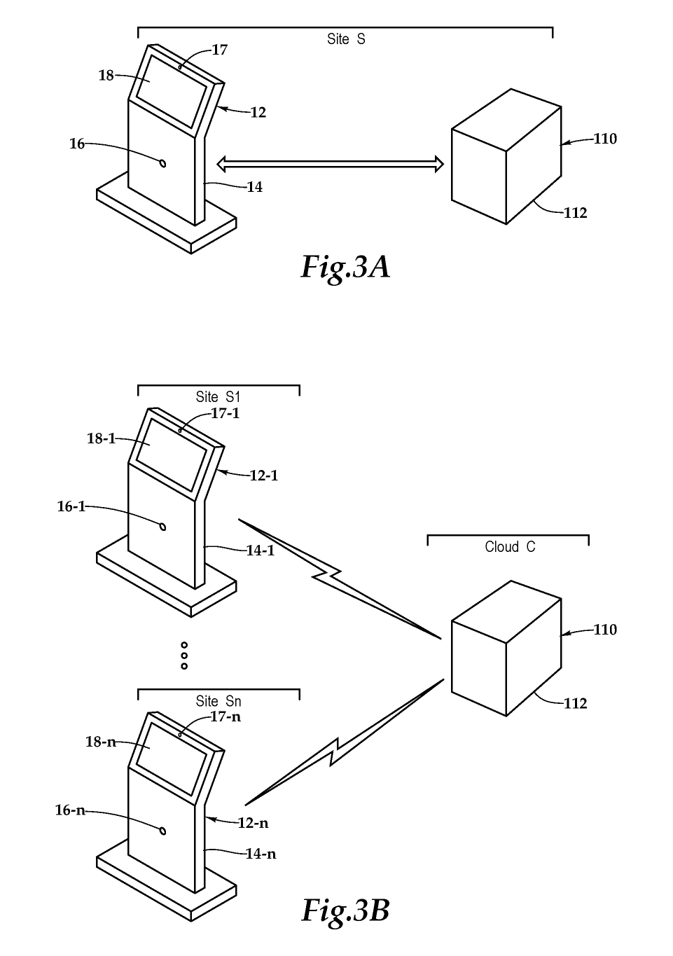

A server 110, which supports the integrated goniometer 12 as part of the integrated goniometry system 10, may be co-located with the integrated goniometer 12 or remotely located to serve multiple integrated goniometers at different sites. Referring now to FIG. 3A, the server 110, which includes a housing 112, is co-located on the site S with the integrated goniometer 12. The server 110 provides various storage and support functionality to the integrated goniometer 12. Referring now to FIG. 3B, the integrated goniometry system 10 may be deployed such that the server 110 is remotely located in the cloud C to service multiple sites S1 . . . Sn with each site having an integrated goniometer 12-1 . . . 12-n and corresponding housings 14-1 . . . 14-n, optical sensing instruments 16-1 . . . 16-n, webcameras 17-1 . . . 17-n, and displays 18-1 . . . 18-n. The server 110 provides various storage and support functionality to the integrated goniometer 12.

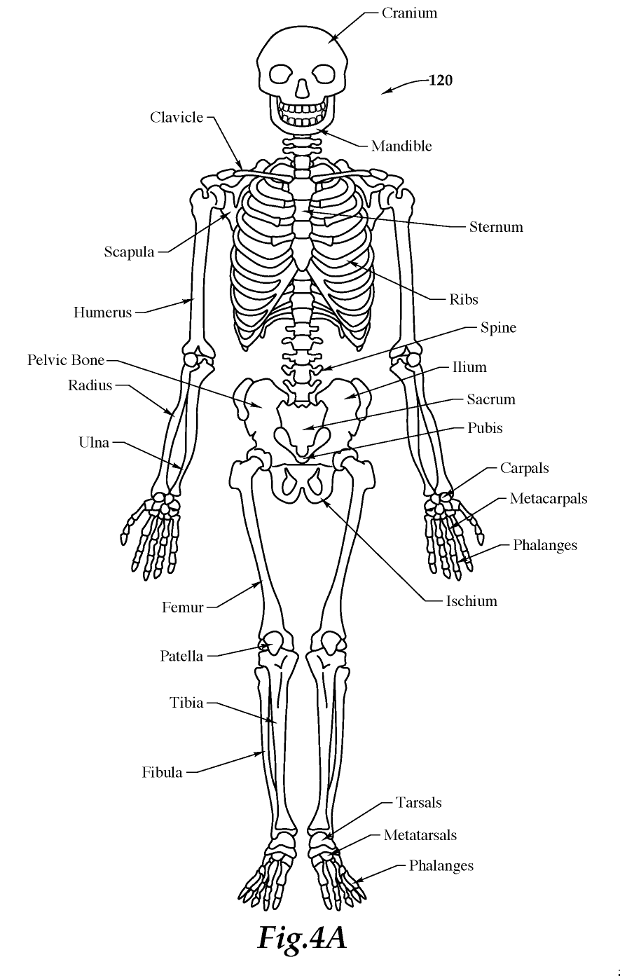

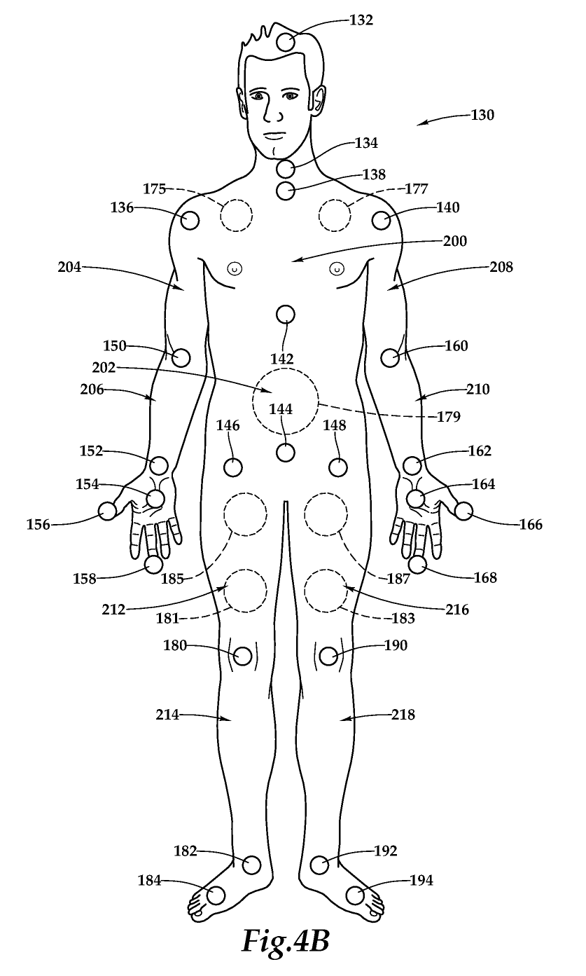

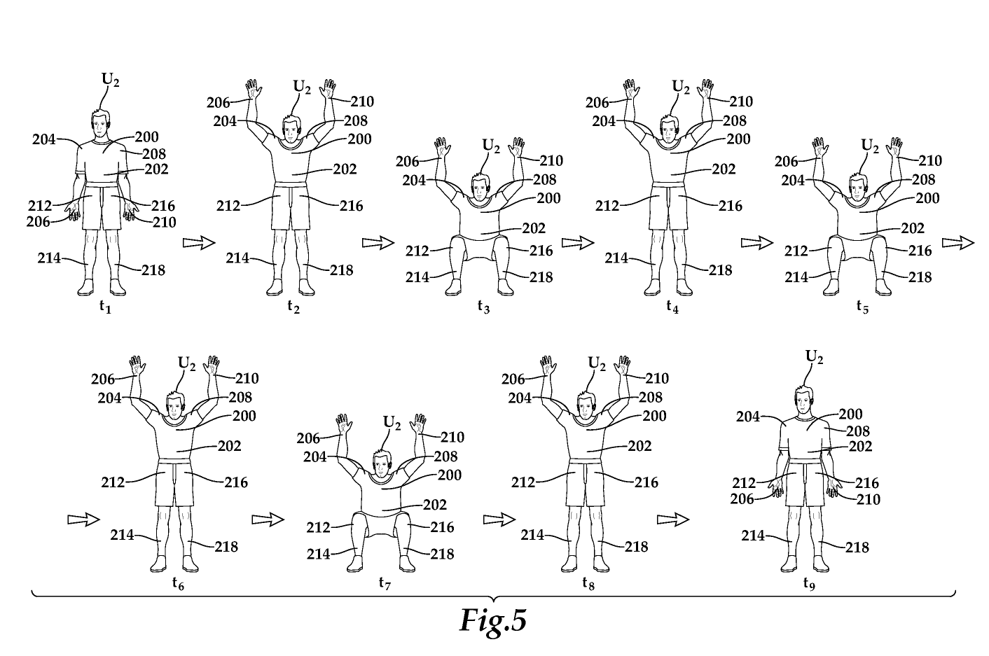

Referring now to FIG. 4A and FIG. 4B, respective embodiments of a human skeleton 120 and body point data 130 captured by the integrated goniometry system 10 are depicted. The body point data 130 approximates certain locations and movements of the human body, represented by the human skeleton 120. More specifically, the body point data 130 is captured by the optical sensing instrument 16 and may include head point data 132, neck point data 134, left shoulder point data 136, spine shoulder point data 138, right shoulder point data 140, spine midpoint point data 142, spine base point data 144, left hip point data 146, right hip point data 148. The body point data 130 may also include left elbow point data 150, left wrist point data 152, left hand point data 154, left thumb point data 156, left hand tip point data 158, right elbow point data 160, right wrist point data 162, right hand point data 164, right thumb point data 166, and right hand tip point data 168. The body point data 130 may also include left knee point data 180, left ankle point data 182, and left foot point data 184, right knee point data 190, right ankle point data 192, and right foot point data 194. It should be appreciated that the body point data 130 may vary depending on application and type of optical sensing instrument selected.

By way of example and not by way of limitation, the body point data 130 may include torso point data 200, torso point data 202, left arm point data 204, left arm point data 206, right arm point data 208, right arm point data 210, left leg point data 212, left leg point data 214, right leg point data 216, and right leg point data 218 for example. In one embodiment, the torso point data 200 or the torso point data 202 may include the left shoulder point data 136, the neck point data 134, the spine shoulder point data 138, the right shoulder point data 140, the spine midpoint data 142, the spine base point data 144, the left hip point data 146, and the right hip point data 148. The left arm point data 204 or the left arm point data 206 may be left elbow point data 150, left wrist point data 152, left hand point data 154, left thumb point data 156, left hand tip point data 158. In some embodiments, the left arm point data 206 may include the left shoulder point data 136. The left leg point data 212 or left leg point data 214 may include the left knee point data 180, the left ankle point data 182, and the left foot point data 184.

The right arm point data 208 or the right arm point data 210 may be the right elbow point data 160, the right wrist point data 162, the right hand point data 164, the right thumb point data 166, or the right hand tip point data 168. In some embodiments, the right arm point data 208 may include the right shoulder point data 140. The right leg point data 216 or right leg point data 218 may include the right knee point data 190, the right ankle point data 192, and the right foot point data 194. Further, it should be appreciated that the torso point data 200, the torso point data 202, the left arm point data 204, the left arm point data 206, the right arm point data 208, the right arm point data 210, the left leg point data 212, the left leg point data 214, the right leg point data 216, and the right leg point data 218 may partially overlap.

Additionally, the body point data 130 captured by the optical sensing instrument 16 may include data relative to locations on the body in the rear of the person or user. This data may be acquired through inference. By way of example, by gathering certain body point data 130 from the front of the person or use, body point data 130 in the rear may be interpolated or extrapolated. By way of example and not by way of limitation, the body point data 130 may include left scap point data 175 and right scap point data 177; torso point data 179; left hamstring point data 181 and right hamstring point data 183; and left glute point data 185 and right glute point data 187. As illustrated and described, the terms “left” and “right” refer to the view of the optical sensing instrument 16. It should be appreciated that in another embodiment the terms “left” and “right” may be used to refer to the left and right of the individual user as well.

In one embodiment, the optical sensing instrument 16 captures the body point data 130 by creating, for each pixel in at least one of the captured image frames, a value representative of a sensor measurement. For example, sensor measurements from each pixel may include the difference in intensity between the pixel in the current frame and those from previous frames, after registering the frames to correct for the displacement of the input images. Additionally, statistical measurements may be made and compared to thresholds indicating the intensity differences over multiple frames. The combined information on intensity differences may be used to identify which pixels represent motion across multiple image frames.

In one embodiment, to detect motion relative to a pixel within an image frame or multiple image frames, the integrated goniometer 12 may determine whether an average difference of the value representative of the sensor measurement of multiple image frames is greater than a scaled average difference and whether the average difference is greater than a noise threshold. The scaled average difference may be determined based on a statistical dispersion of data resulting from normalizing the difference of the value representative of the sensor measurement of a pixel of the plurality of image frames and sensor noise, registration accuracy, and changes in the image from frame-to-frame such as rotation, scale and perspective. The noise threshold may be determined based on measured image noise and the type of optical sensing instrument providing the body point data 130.

As previously discussed, the integrated goniometry system 10 performs measurement and scoring of physiology. In one embodiment, measurements during repetitions of an exercise movement, such as three squats, are recorded over domains of mobility, activation, posture, and symmetry. It should be appreciated that although the exercise movement is presented as a squat, other exercise movements are within the teachings presented herein. Mobility may be the range of motion achieved in key joints, such as the elbow (Humerus at Ulna), shoulder (Clavicle at Humerus), hip (Pelvic bone at the Femur), and knee (Patella). Activation may be the ability to control and maintain optimal position and alignment for glute (inferred from data collected near the Pelvic Bone and Femur), scap (inferred from data collected near the Clavicle), and squat depth (inferred from data collected near the Pelvic Bone and Femur). Posture may be the static alignment while standing normally for the shoulder (Clavicle at Humerus), hip (Pelvic bone at the Femur), valgus (oblique displacement of the Patella during the exercise movement), backbend, and center of gravity. Symmetry may be the imbalance between right and left sides during movement of the elbow (Humerus at Ulna), shoulder (Clavicle at Humerus), knee (Patella), squat depth, hip (Pelvic bone at the Femur), and center of gravity.

Mobility may relate to the angle of the joint and be measured in each video frame. With respect to the elbow, the left arm point data 204, 206 or the right arm point data 208, 210 may be utilized to capture the average angle. With respect to the shoulder, the torso point data 200, 202, and the left arm point data 204, 206 or the right arm point data 208, 210 may be utilized to capture the average angle. With respect to the hip, the torso point data 200, 202 and the left arm point data 204, 206 or the right arm point data 208, 210 may be utilized to capture the average maximum angle. With respect to the knee, the left leg point data 212, 214 or the right leg point data 216, 218 may be utilized.

Activation may relate to the averaged position of joints for each repetition of the exercise movement. The glute may reflect an outward knee movement. A reference point may be created by sampling multiple frames before any exercise trigger and any movement is detected. From these multiple frames, an average start position of the knee may be created. After the exercise trigger, the displacement of the knee is compared to the original position and the values are then averaged over the repetitions of the exercise movement. The left leg point data 212, 214 and the right leg point data 216, 218 may be utilized for scoring activation.

Posture may relate to the difference between the ground to joint distance of each side while standing still. Similar to the approach with mobility and activation, selected frames of body point data collected by the integrated goniometer 12 may be averaged. Shoulder, hip, xiphoid process, valgus as measured by the knee. Backbend (forward spine angle relative to the ground) may be measured. With respect to the shoulder, the torso point data 200, 202, and the left arm point data 204, 206 or the right arm point data 208, 210 may be utilized. With respect to the hip, the torso point data 200, 202 and the left arm point data 204, 206 or the right arm point data 208, 210 may be utilized. With respect to the knee, the left leg point data 212, 214 or the right leg point data 216, 218 may be utilized. With respect to the valgus, the torso point data 200, 202 and the left leg point data 212, 214 or the right leg point data 216, 218 may be utilized. Symmetry may relate to an asymmetry index, known as AI %, for various measures including left and right elbow; left and right shoulder; left and right knee; left and right femur angles; left and right hip flexion; and the center of gravity as measured by the position of the xiphoid process relative to the midpoint. Various combinations of the torso point data 200, 202, the left arm point data 204, 206, the right arm point data 208, 210, the left leg point data 212, 214, and the right leg point data 216, 218 may be utilized to capture the necessary body point data for the symmetry measurements.

Referring now to FIG. 5, body point data 130 associated with a set number of repetitions of an exercise movement by the user U₂ are monitored and captured by the integrated goniometry system 10. As shown, in the illustrated embodiment, the user U₂ executes three squats and specifically three bodyweight overhead squats at t₃, t₅, and t₇. It should be understood, however, that a different number of repetitions may be utilized and is within the teachings presented herein. At t₁ and t₉, user U₂ is at a neutral position, which may be detected by sensing the body point data 130 within the virtual volumetric cubic area 28 of the stage 24 or at t₉, an exercise end position which is sensed with the torso point data 200, 202 in an upright position superposed above the left leg point data 212, 214 and the right leg point data 216, 218 with the left arm point data 204, 206 and right arm point data 208, 210 laterally offset to the first torso point data and second torso point data.

At t₂, t₄, t₆, and t₈, user U₂ is at an exercise start position. The exercise start position may be detected by the torso point data 200, 202 in an upright position superposed above the left leg point data 212, 214 and the right leg point data 216, 218 with the left arm point data 204, 206 and the right arm point data 208, 210 superposed above the torso point data 200, 202. From an exercise start position, the user U₂ begins a squat with an exercise trigger. During the squat or other exercise movement, the body point data 130 is collected. The exercise trigger may be displacement of the user from the exercise start position by sensing displacement of the body point data 130. Each repetition of the exercise movement, such as a squat, may be detected by sensing body point data 130 returning to its position corresponding to the exercise start position. By way of example, the spine midpoint point data 142 may be monitored to determine to mark the completion of exercise movement repetitions.

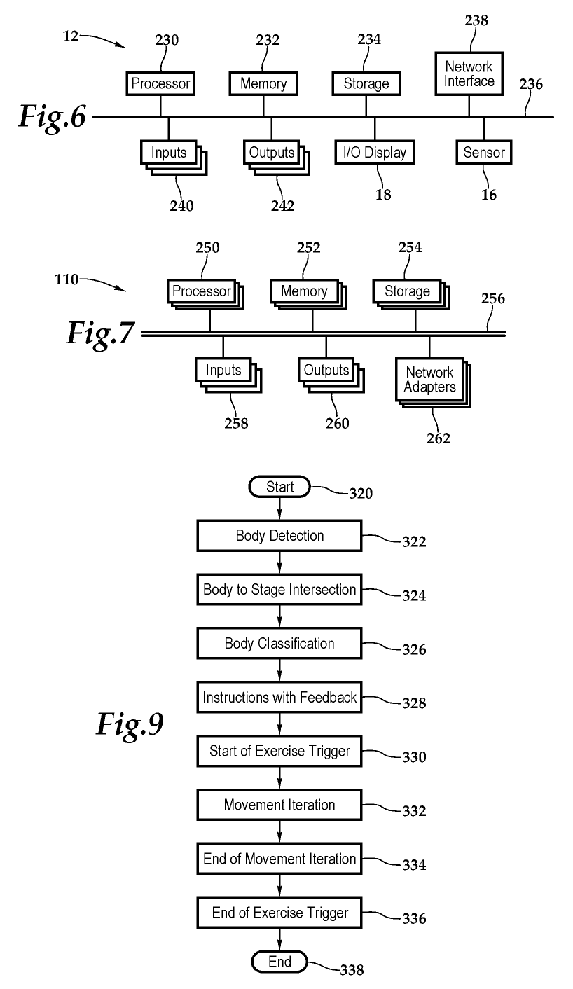

Referring to FIG. 6, within the housing 14 of the integrated goniometer 12, a processor 230, memory 232, and storage 234 are interconnected by a bus architecture 236 within a mounting architecture that also interconnects a network interface 238, inputs 240, outputs 242, the display 18, and the optical sensing instrument 16. The processor 230 may process instructions for execution within the integrated goniometer 12 as a computing device, including instructions stored in the memory 232 or in storage 234. The memory 232 stores information within the computing device. In one implementation, the memory 232 is a volatile memory unit or units. In another implementation, the memory 232 is a non-volatile memory unit or units. Storage 234 provides capacity that is capable of providing mass storage for the integrated goniometer 12. The network interface 238 may provide a point of interconnection, either wired or wireless, between the integrated goniometer 12 and a private or public network, such as the Internet. Various inputs 240 and outputs 242 provide connections to and from the computing device, wherein the inputs 240 are the signals or data received by the integrated goniometer 12, and the outputs 242 are the signals or data sent from the integrated goniometer 12. The display 18 may be an electronic device for the visual presentation of data and may, as shown in FIG. 6, be an input/output display providing touchscreen control. The optical sensing instrument 16 may be a camera, a kinetic camera, a point-cloud camera, a laser-scanning camera, a high definition video camera, an infrared sensor, or an RGB composite camera, for example.

The memory 232 and storage 234 are accessible to the processor 230 and include processor-executable instructions that, when executed, cause the processor 230 to execute a series of operations. The processor-executable instructions cause the processor 230 to display an invitation prompt on the interactive portal. The invitation prompt provides an invitation to the user to enter the stage prior to the processor-executable instructions causing the processor 230 to detect the user on the stage by sensing body point data 130 within the virtual volumetric cubic area 28. By way of example and not by way of limitation, the body point data 130 may include first torso point data, second torso point data, first left arm point data, second left arm point data, first right arm point data, second right arm point data, first left leg point data, second left leg point data, first right leg point data, and second right leg point data, for example.

The processor-executable instructions cause the processor 230 to display an exercise movement prompt 60 on the interactive portal 20. The exercise movement prompt 60 provides instructions for the user to execute an exercise movement for a set number of repetitions with each repetition being complete when the user returns to an exercise start position. The processor 230 is caused by the processor-executable instructions to detect an exercise trigger. The exercise trigger may be displacement of the user from the exercise start position by sensing displacement of the related body point data 130. The processor-executable instructions also cause the processor 230 to display an exercise end prompt on the interactive portal 20. The exercise end prompt provides instructions for the user to stand in an exercise end position. Thereafter, the processor 230 is caused to detect the user standing in the exercise end position.

The processor-executable instructions cause the processor 230 to calculate one or more of several scores including calculating a mobility score by assessing angles using the body point data 130, calculating an activation score by assessing position within the body point data 130, calculating a posture score by assessing vertical differentials within the body point data 130, and calculating a symmetry score by assessing imbalances within the body point data 130. The processor-executable instructions may also cause the processor 230 to calculate a composite score 88 based on one or more of the mobility score 80, the activation score 82, the posture score 84, or the symmetry score 86. The processor-executable instructions may also cause the processor 230 to determine an exercise recommendation based on one or more of the composite score 88, the mobility score 80 the activation score 82, the posture score 84, or the symmetry score 86.

Referring now to FIG. 7, one embodiment of the server 110 as a computing device includes, within the housing 112, a processor 250, memory 252, storage 254, interconnected with various buses 256 in a common or distributed, for example, mounting architecture, that also interconnects various inputs 258, various outputs 260, and network adapters 262. In other implementations, in the computing device, multiple processors and/or multiple buses may be used, as appropriate, along with multiple memories and types of memory. Further still, in other implementations, multiple computing devices may be provided and operations distributed therebetween. The processor 250 may process instructions for execution within the server 110, including instructions stored in the memory 252 or in storage 254. The memory 252 stores information within the server 110 as the computing device. In one implementation, the memory 252 is a volatile memory unit or units. In another implementation, the memory 252 is a non-volatile memory unit or units. Storage 254 includes capacity that is capable of providing mass storage for the server 110. Various inputs 258 and outputs 260 provide connections to and from the server 110, wherein the inputs 258 are the signals or data received by the server 110, and the outputs 260 are the signals or data sent from the server 110. The network adapters 262 connect the server 110 to a network shared by the integrated goniometer 12.

The memory 252 is accessible to the processor 250 and includes processor-executable instructions that, when executed, cause the processor 250 to execute a series of operations. The processor-executable instructions cause the processor 250 to update periodically or on-demand, depending on the operational configuration, a database which may be part of storage 254 of body point data, exercise recommendations, composite scores, mobility scores, activation scores, posture scores, and symmetry scores associated with various users. The processor-executable instructions cause the processor 250 to make this database or a portion thereof available to the integrated goniometer by way of the integrated goniometer 12 receiving the information through fetching or the server 110 sending the requested information. Further, the processor-executable instructions cause the processor 250 to execute any of the processor-executable instructions presented in association with the integrated goniometer 12, for example.

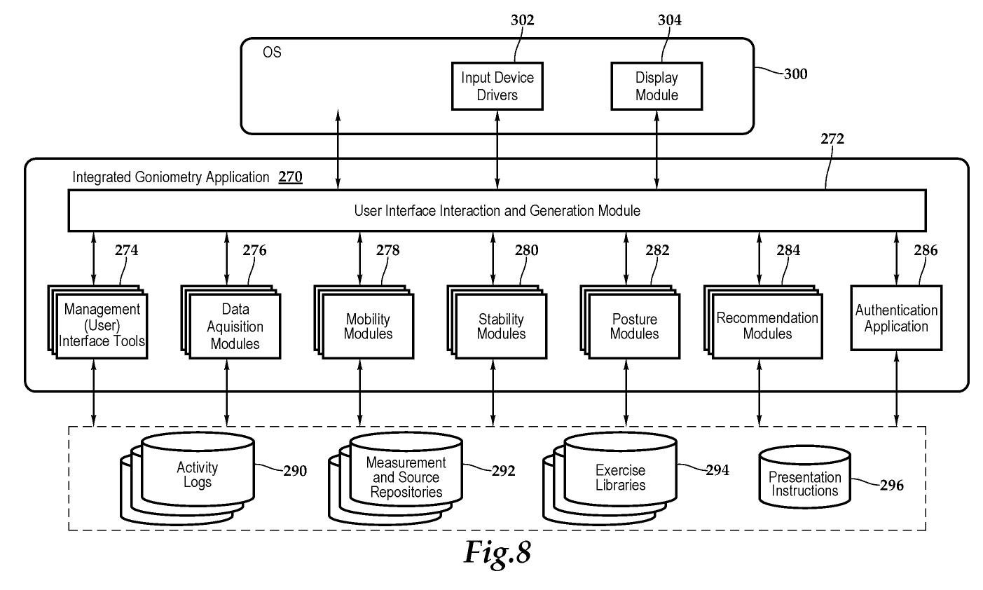

FIG. 8 conceptually illustrates the software architecture of an integrated goniometry application 270 of some embodiments that may automate the biomechanical evaluation process and provide recommended exercises to improve physiological inefficiencies of a user. In some embodiments, the integrated goniometry application 270 is a stand-alone application or is integrated into another application, while in other embodiments the application might be implemented within an operating system 300. Furthermore, in some embodiments, the integrated goniometry application 270 is provided as part of a server-based solution or a cloud-based solution. In some such embodiments, the integrated goniometry application 270 is provided via a thin client. That is, the integrated goniometry application 270 runs on a server while a user interacts with the application via a separate machine remote from the server. In other such embodiments, integrated goniometry application 270 is provided via a thick client. That is, the integrated goniometry application 270 is distributed from the server to the client machine and runs on the client machine.

The integrated goniometry application 270 includes a user interface (UI) interaction and generation module 272, management (user) interface tools 274, data acquisition modules 276, mobility modules 278, stability modules 280, posture modules 282, recommendation modules 284, and an authentication application 286. The integrated goniometry application 270 has access to, activity logs 290, measurement and source repositories 292, exercise libraries 294, and presentation instructions 296, which presents instructions for the operation of the integrated goniometry application 270 and particularly, for example, the aforementioned interactive portal 20 on the display 18. In some embodiments, storages 290, 292, 294, and 296 are all stored in one physical storage. In other embodiments, the storages 290, 292, 294, and 296 are in separate physical storages, or one of the storages is in one physical storage while the other is in a different physical storage.

The UI interaction and generation module 272 generates a user interface that allows, through the use of prompts, the user to quickly and efficiently perform a set of exercise movements to be monitored with the body point data 130 collected from the monitoring furnishing an automated biomechanical movement assessment scoring and related recommended exercises to mitigate inefficiencies. Prior to the generation of automated biomechanical movement assessment scoring and related recommended exercises, the data acquisition modules 276 may be executed to obtain instances of the body point data 130 via the optical sensing instrument 16. Following the collection of the body point data 130, the mobility modules 278, stability modules 280, and the posture modules 282 are utilized to determine a mobility score 80, an activation score, and a posture score 84, for example. More specifically, in one embodiment, the mobility modules 278 measure a user's ability to freely move a joint without resistance. The stability modules 280 provide an indication of whether a joint or muscle group may be stable or unstable. The posture modules 282 may provide an indication of physiological stresses presented during a natural standing position. Following the assessments and calculations by the mobility modules 278, stability modules 280, and the posture modules 282, the recommendation modules 284 may provide a composite score 88 based on the mobility score 80, the activation score, and the posture score 84 as well as exercise recommendations for the user. The authentication application 286 enables a user to maintain an account, including an activity log and data, with interactions therewith.

In the illustrated embodiment, FIG. 8 also includes the operating system 300 that includes input device drivers 302 and a display module 304. In some embodiments, as illustrated, the input device drivers 302 and display module 304 are part of the operating system 300 even when the integrated goniometry application 270 is an application separate from the operating system 300. The input device drivers 302 may include drivers for translating signals from a keyboard, a touch screen, or an optical sensing instrument, for example. A user interacts with one or more of these input devices, which send signals to their corresponding device driver. The device driver then translates the signals into user input data that is provided to the UI interaction and generation module 272.

FIG. 9 depicts one embodiment of a method for integrated goniometric analysis. At block 320, the methodology begins with the integrated goniometer positioned facing the stage. At block 322, multiple bodies are simultaneously detected by the integrated goniometer in and around the stage. As the multiple bodies are detected, a prompt displayed on the interactive portal of integrated goniometer invites one of the individuals to the area of the stage in front of the integrated goniometer. At block 324, one of the multiple bodies is isolated by the integrated goniometer and identified as an object of interest once it separates from the group of multiple bodies and enters the stage in front of the integrated goniometer. At block 326, the identified body, a user, is tracked as a body of interest by the integrated goniometer.

At block 328, the user is prompted to position himself into the appropriate start position which will enable the collection of a baseline measurement and key movement measurements during exercise. At this point in the methodology, the user is prompted by the integrated goniometer to perform the exercise start position and begin a set repetitions of an exercise movement. The integrated goniometer collects body point data 130 to record joint angles and positions. At block 330, the integrated goniometer detects an exercise trigger which is indicative of phase movement discrimination being performed in a manner that is independent of the body height, width, size or shape or the user.

At block 332, the user is prompted by the integrated goniometer to repeat the exercise movement as repeated measurements provide more accurate and representative measurements. A repetition is complete when the body of the user returns to the exercise start position. The user is provided a prompt to indicate when the user has completed sufficient repetitions of the exercise movement. With each repetition, once in motion, monitoring of body movement will be interpreted to determine a maximum, minimum, and moving average for the direction of movement, range of motion, depth of movement, speed of movement, rate of change of movement, and change in the direction of movement, for example. At block 334, the repetitions of the exercise movement are complete. At block 336, once the required number of repetitions of the exercise movement are complete, the user is prompted to perform an exercise end position, which is a neutral pose. With the exercise movements complete, the integrated goniometry system begins calculating results and providing the results and any exercise recommendations to the user.

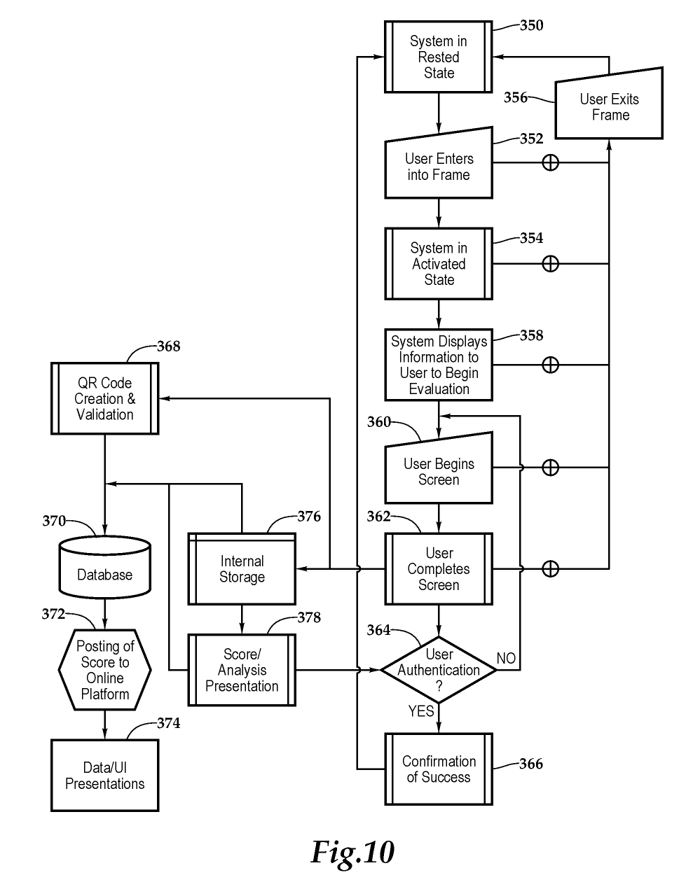

FIG. 10 shows how the user U₂ of FIG. 5, for example, may begin and end a musculoskeletal evaluation in accordance with aspects of the present disclosure. For example, at subroutine block 350, upon launching the application by opening an executable file, the musculoskeletal evaluation system of the integrated goniometer may remain in a “rested” state, and the optical sensing instrument is not processing any data. At summoning junction block 352, in response to the detection of an entry of the user U₂ into its field of view, the optical sensing instrument 16 may be activated to start recording user motion data and advance to a subroutine block 354. However, if the user the optical sensing instrument has been detected to exit the optical sensing instrument's field of view at summoning junction block 356, the system may return to its “rested” state. Once the system is “active” at the subroutine block 354, there may be a prompt in the form of a transitional animation that launches a live video feed on the display of the integrated goniometer, which may provide the user U₂ with on-screen instructions. That is, in one embodiment, at process block 358, the display module may be configured to provide clear and detailed instructions to the user U₂ on how to begin the evaluation. These instructions may include at least one of: animation showing how to perform the exercise movement; written detailed instructions on how to perform the exercise movement; written instructions on how to progress and begin the evaluation movement; audio detailed instructions on how to perform the exercise movement; and audio instructions on how to progress and begin the evaluation movement.

At summoning junction block 360, following the instructions provided on screen, as an example, the user U₂ may face the display and keep the user's feet pointed forward at shoulder width apart. The system may confirm that the user U₂ is in a correct position and prompt her to, e.g., raise her hands or begin any suitable user movement for musculoskeletal evaluation purposes. A countdown may begin for the user U₂ to perform a series of specified movements, such as three overhead squats. Upon completion at subroutine block 362, the user U₂ may be prompted to return to a rested state such as lowering her hands, thereby ending the evaluation.

Following the completion of the exercise movements, the identity of the user U₂ is created or validated at subroutine block 368 prior to the identity being stored at database block 370 prior to, in one embodiment, posting of the user's scores online at posting block 372 with the user's scores being accessible by way of a data and user interface at user action block 374. Regarding the user's scores, returning to subroutine block 362, following the completion of the exercise movements, the body point data 130 collected by the integrated goniometer 12 is stored at internal storage block 376 prior to analysis at subroutine block 378, which results in storage at database block 370 and upon completion of the user authentication at decision block 364, presentation of the results, including any exercise recommendations at successful completion at subroutine block 366.

The order of execution or performance of the methods and data flows illustrated and described herein is not essential, unless otherwise specified. That is, elements of the methods and data flows may be performed in any order, unless otherwise specified, and that the methods may include more or less elements than those disclosed herein. For example, it is contemplated that executing or performing a particular element before, contemporaneously with, or after another element are all possible sequences of execution.

While this invention has been described with reference to illustrative embodiments, this description is not intended to be construed in a limiting sense. Various modifications and combinations of the illustrative embodiments as well as other embodiments of the invention, will be apparent to persons skilled in the art upon reference to the description. It is, therefore, intended that the appended claims encompass any such modifications or embodiments.

What is claimed is:

1. An integrated goniometry system comprising:
a housing securing an optical sensing instrument, a display, a processor, and memory therewith;
a busing architecture communicatively interconnecting the sensor, the display, the processor, and the memory;
the optical sensing instrument monitoring a stage, the stage being a virtual volumetric cubic area that is compatible with human exercise positions and movement, the stage including a rectangular volume in space at a monitoring distance from the sensor;
the display facing the stage, the display includes an interactive portal; and
the memory accessible to the processor, the memory including processor-executable instructions that, when executed, cause the processor to:
sense body point data of a user with the optical sensing instrument when the user is on the stage, the body point data including a first torso point data, a second torso point data, a left arm point data, a right arm point data, a left leg point data, and a right leg point data,
display an instruction prompt on the interactive portal, the instruction prompt providing instructions for the user to stand in the baseline position,
detect the user in the baseline position by sensing the first torso point data and second torso point data in an upright position superposed above the left leg point data and the right leg point data with the left arm point data and right arm point data laterally offset to the first torso point data and second torso point data,
display an exercise prepare prompt on the interactive portal, the exercise prepare prompt providing instructions for the user to stand in the exercise start position,
detect the user in the exercise start position by sensing the first torso point data and second torso point data in an upright position superposed above the left leg point data and the right leg point data with the left arm point data and right arm point data superposed above the first torso point data and second torso point data,
display an exercise movement prompt on the interactive portal, the exercise movement prompt providing instructions for the user to execute an exercise movement for a set number of repetitions, each repetition being complete when the user returns to the exercise start position, and
detect an exercise trigger, the exercise trigger being displacement of the user from the exercise start position by sensing displacement of the body point data.

2. The integrated goniometry system as recited in claim 1, wherein the processor-executable instructions further comprise instructions that, when executed, cause the processor to:
display an invitation prompt on the interactive portal, the invitation prompt providing an invitation to the user to enter the stage, and
detect the user on the stage by sensing the body point data within the virtual volumetric cubic area.

3. The integrated goniometry system as recited in claim 1, wherein the processor-executable instructions further comprise instructions that, when executed, cause the processor to:
display an exercise end prompt on the interactive portal, the exercise end prompt providing instructions for the user to stand in an exercise end position, and
detect the user standing in the exercise end position by sensing the first torso point data and second torso point data in an upright position superposed above the left leg point data and the right leg point data with the left arm point data and right arm point data laterally offset to the first torso point data and second torso point data.

4. The integrated goniometry system as recited in claim 1, wherein the optical sensing instrument further comprises an instrument selected from the group consisting of a camera, a kinetic camera, a point-cloud camera, a laser-scanning camera, a high definition video camera, an infrared sensor, and an RGB composite camera.

5. The integrated goniometry system as recited in claim 1, wherein the exercise movement further comprises a squat.

6. The integrated goniometry system as recited in claim 1, wherein the first torso point data comprises point data selected from the group consisting of shoulder left point data, neck point data, spine shoulder point data, shoulder right point data, spine midpoint data, spine base point data, left hip point data, and right hip point data.

7. The integrated goniometry system as recited in claim 1, wherein the left arm point comprises point data selected from the group consisting of left elbow point data, left wrist point data, left hand point data, left thumb point data, left hand tip point data.

8. The integrated goniometry system as recited in claim 1, wherein the left leg point data comprises point data selected from the group consisting of left knee point data, left ankle point data, and left foot point data.

9. An integrated goniometry system comprising:
a housing securing an optical sensing instrument, a display, a processor, and memory therewith;
a busing architecture communicatively interconnecting the sensor, the display, the processor, and the memory;
the optical sensing instrument monitoring a stage, the stage being a virtual volumetric cubic area that is compatible with human exercise positions and movement, the stage including a rectangular volume in space at a monitoring distance from the sensor;
the display facing the stage, the display includes an interactive portal; and
the memory accessible to the processor, the memory including processor-executable instructions that, when executed, cause the processor to:
sense body point data of a user with the optical sensing instrument when the user is on the stage, the body point data including first torso point data, second torso point data, first left arm point data, second left arm point data, first right arm point data, second right arm point data, first left leg point data, second left leg point data, first right leg point data, and second right leg point data,
display an exercise movement prompt on the interactive portal, the exercise movement prompt providing instructions for the user to execute an exercise movement for a set number of repetitions, each repetition being complete when the user returns to an exercise start position,
detect an exercise trigger, the exercise trigger being displacement of the user from the exercise start position by sensing displacement of the body point data, the exercise start position being the first torso point data and second torso point data in an upright position superposed above the first and second left leg point data and the first and second right leg point data with the first and second left arm point data and the first and second right arm point data superposed above the upper torso point data and lower torso point data, and
calculate a mobility score by assessing angles using the body point data.

10. The integrated goniometry system as recited in claim 9, wherein the processor-executable instructions further comprise instructions that, when executed, cause the processor to:
calculate an activation score by assessing position of the body point data.

11. The integrated goniometry system as recited in claim 10, wherein the processor-executable instructions further comprise instructions that, when executed, cause the processor to:
calculate a posture score by assessing vertical differentials within the body point data.

12. The integrated goniometry system as recited in claim 11, wherein the processor-executable instructions further comprise instructions that, when executed, cause the processor to:
calculate a symmetry score by assessing imbalances within the body point data.

13. The integrated goniometry system as recited in claim 9, wherein the optical sensing instrument further comprises an instrument selected from the group consisting of a camera, a kinetic camera, a point-cloud camera, a laser-scanning camera, a high definition video camera, an infrared sensor, and an RGB composite camera.

14. The integrated goniometry system as recited in claim 9, wherein the exercise movement further comprises a squat.

15. The integrated goniometry system as recited in claim 9, wherein the first torso point data and the second torso point data each comprise point data selected from the group consisting of shoulder left point, neck point, spine shoulder point, shoulder right point, spine midpoint, spine base point, left hip point, and right hip point.

16. The integrated goniometry system as recited in claim 9, wherein the first left arm point comprises point data selected from the group consisting of left elbow point data, left wrist point data, left hand point data, left thumb point data, and left hand tip point data.

17. The integrated goniometry system as recited in claim 9, wherein the first left leg point comprises point data selected from the group consisting of left knee point data, left ankle point data, and left foot point data.

18. An integrated goniometry system comprising:
a housing securing an optical sensing instrument, a display, a processor, and memory therewith;
a busing architecture communicatively interconnecting the sensor, the display, the processor, and the memory;
the optical sensing instrument monitoring a stage, the stage being a virtual volumetric cubic area that is compatible with human exercise positions and movement, the stage including a rectangular volume in space at a monitoring distance from the sensor;
the display facing the stage, the display includes an interactive portal; and
the memory accessible to the processor, the memory including processor-executable instructions that, when executed, cause the processor to:
display an exercise movement prompt on the interactive portal, the exercise movement prompt providing instructions for a user on the stage to execute a set number of repetitions of a bodyweight overhead squat,
sense body point data of the user with the optical sensing instrument during each bodyweight overhead squat, the body point data including first torso point data, second torso point, first left arm point data, second left arm point data, first right arm point data, second right arm point data, first left leg point data, second left leg point data, first right leg point data, and second right leg point data, and
calculate a mobility score by assessing angles within the body point data.

19. The integrated goniometry system as recited in claim 18, wherein the processor-executable instructions further comprise instructions that, when executed, cause the processor to:
calculate an activation score by assessing position within the body point data,
calculate a posture score by assessing vertical differentials within the body point data, and
calculate a symmetry score by assessing imbalances within the body point data.

20. The integrated goniometry system as recited in claim 18, wherein the optical sensing instrument further comprises an instrument selected from the group consisting of a camera, a kinetic camera, a point-cloud camera, a laser-scanning camera, a high definition video camera, an infrared sensor, and an RGB composite camera.
